(19) United States
(12) Patent Application Publication
(10) Pub. No.: US 2008/0312482 A1
(43) Pub. Date: Dec. 18, 2008

(54) Process for para-xylene production from 2,4,4-trimethylpentene

(76) Inventors: Deng-Yang Jan, Elk Grove Village, IL (US); Stanley J. Frey, Palatine, IL (US); Jaime G. Moscoso, Mount Prospect, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/027,548

(22) Filed: Dec. 30, 2004

(51) Int. Cl. C07C 2/52

(52) U.S. Cl. 585/418

(57) ABSTRACT

The subject process obtains a high yield of high-purity para-xylene from 2,4,4-trimethylpentene as contained in butene dimer. A process combination may include dimerization of the isobutene to obtain C₈ iso-olefins and isoparaffins, aromatization of the dimerized C₈ product, and recovery of high-purity para-xylene from the dimerized product by low-intensity crystallization. Aromatization is effected using a catalyst comprising a large-pore molecular sieve. Each of the processing steps may be tailored to the overall objective of high para-xylene yield from a relatively inexpensive feedstock.

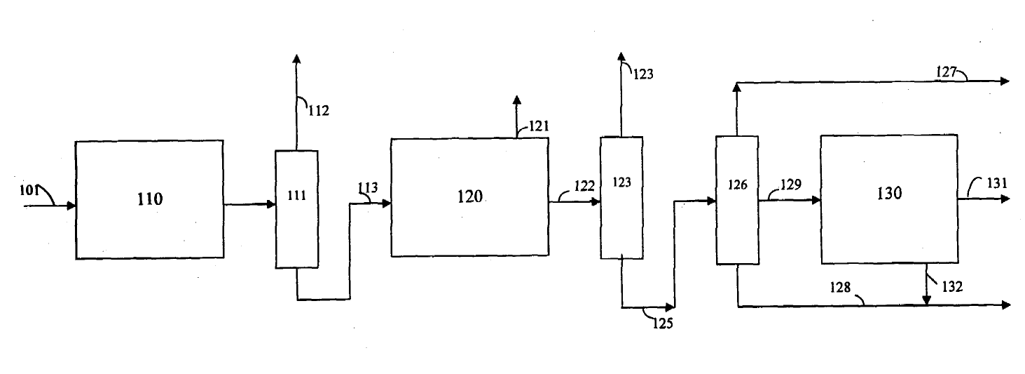

### FIELD OF THE INVENTION

The present invention relates to the field of aromatic petrochemicals. More specifically, the invention relates to the production of para-xylene from aliphatic hydrocarbons.

### BACKGROUND OF THE INVENTION

Para-xylene is an important intermediate in the chemical and fiber industries. Terephthalic acid derived from para-xylene is used to produce polyester fabrics and other articles which are in wide use today.

Usually para-xylene is produced, in a series of steps, from naphtha fractionated from crude oil. Naphtha is hydrotreated and reformed to yield aromatics, which then are fractionated to separate typically benzene, toluene and C₈ aromatics comprising xylenes from C₉ and heavier aromatics. Toluene and C₉ aromatics may be disproportionated to yield additional xylenes. Xylene isomers, with the usual priority being para-xylene, are separated from the mixed C₈-aromatics stream using one or a combination of adsorptive separation, crystallization and fractional distillation, with adsorptive separation being most widely used in newer installations for para-xylene production. Other C₈ isomers may be isomerized and returned to the separation unit to yield additional para-xylene.

In the Journal of Catalysis 1 (1962), pp. 313-328, Pines and Csicsery disclose the aromatization of trimethylpentanes to xylenes, using a nonacidic chromia-alumina catalyst; 2,2,4-trimethylpentane formed only para-xylene. In the proceedings of the 1962 Radioisotopes Physical Science Industrial Process Conference at pages 205-216, Cannings et al. teach dehydrocyclization of 2,2,4-trimethylpentane over a potassium- and cerium-promoted chromia-alumina catalyst to selectively yield para-xylene. British Patent 795,235 teaches the manufacture of para-xylene from 2,4,4-trimethylpentene using a catalyst comprising a Group VI-A oxide, exemplified as a series of chromia-containing catalysts. U.S. Pat. No. 3,202,725 discloses dehydrogenation of isobutane and recycle di-isobutylene using a chromia-alumina catalyst to yield para-xylene and isobutene, plus dimerization of the isobutene using a silica-alumina, phosphoric acid or sulfuric acid catalyst to yield primarily di-isobutylene recycle. U.S. Pat. No. 3,462,505 discloses the dehydrocyclization of 2,2,4-trimethylpentane to yield para-xylene using a catalyst comprising chromia, magnesia and an alkali metal on activated alumina. U.S. Pat. No. 3,766,291 discloses disproportionation of amylene to 2,5-dimethylhexene, which then is selectively converted to para-xylene over a catalyst comprising a Group II metal (exemplified by Zn) aluminate, tin-group metal, and Group VIII metal. U.S. Pat. No. 4,910,357 teaches the aromatization of dimethylhexanes, especially those contained in alkylate, using a catalyst comprising a dehydrogenation metal and a nonacidic crystalline support containing Sn, Tl, In and/or Pb. U.S. Pat. No. 6,177,601 B1 teaches aromatization of 2,5-dimethylhexane to selectively produce para-xylene, using a nonacidic L-zeolite catalyst. U.S. Publication 2004/0044261A1 teaches production of para-xylene from a feedstock rich in C₈ isoalkanes or isoalkenes using a catalyst comprising a molecular sieve, Group VIII metal and two or more of Si, Al, P, Ge, Ga and Ti. U.S. Publication 2004/0015026 discloses the manufacture of para-xylene from 2,2,4-trimethylpentane using a catalyst comprising chromium. It should be noted that chromium, as a catalyst constituent, is a toxic element.

None of the above references discloses the selective process of the present invention. The art has not heretofore recognized how to overcome the difficulty of selectively converting 2,4,4-trimethylpentene to para-xylene.

### BRIEF DESCRIPTION OF THE INVENTION

In a broad embodiment this invention is a process for the production of para-xylene from 2,4,4-trimethylpentene. The process converts a butene dimer comprising 2,4,4-trimethylpentene in an aromatization zone by contact with a nonacidic zeolitic aromatization catalyst at aromatization conditions to obtain a high yield of para-xylene.

In a specific embodiment this invention is a process combination for the production of para-xylene from 2,4,4-trimethylpentene. The process converts a butene dimer comprising 2,4,4-trimethylpentene in an aromatization zone by contact with a nonacidic zeolitic aromatization catalyst at aromatization conditions to obtain a high yield of para-xylene. Effluent from the aromatization zone is fractionated to recover mixed C₈ aromatics having a high concentration of para-xylene, which pass to a separation zone for separation of para-xylene from residual C₈ aromatics. Preferably the separation comprises a single-stage crystallizer.

In a more specific embodiment, this invention comprises processing an isobutene-containing stream in a dimerization zone. In the dimerization zone, the isobutene-containing stream contacts a dimerization catalyst at dimerization conditions to recover a butene dimer comprising 2,4,4-trimethylpentene. The butene dimer passes to an aromatization zone in which 2,4,4-trimethylpentene is converted to obtain a high yield of para-xylene by contact with an aromatization catalyst at aromatization conditions. Effluent from the aromatization zone is fractionated to recover mixed C₈ aromatics which pass to a separation zone for separation of para-xylene from residual C₈ aromatics. Preferably the separation comprises a single-stage crystallizer.

The process of the invention effectively converts 2,4,4-trimethylpentene to obtain a high yield of valuable para-xylene.

### BRIEF DESCRIPTION OF THE DRAWINGS

The FIGURE is a schematic process flow diagram of the invention when processing an isobutene concentrate to yield para-xylene.

### DETAILED DESCRIPTION OF THE INVENTION

A process combination and individual operational steps are described in conjunction with the FIGURE. The FIGURE shows only those portions of the process that are necessary to gain an understanding of the invention and the means of integrating the different process steps that comprise the invention. Further details related to heaters, coolers, exchangers, valves, control means, pumps, compressors, and other necessary processing equipment are well known to those skilled in the art and not described in detail unless necessary for an understanding of the invention. Also, this description does not exclude from the inventive concept other embodiments which may result from the modification of the descriptions by a skilled routineer.

The FIGURE illustrates the process combination of the invention when processing a feed rich in isobutene. In the dimerization zone 110, the isobutene-rich feed 101 passes through a series of dimerization reactors. Zone 110 is divided into multiple reactor stages in order that the dimerization reaction temperature can be controlled by injection of quench between stages. The isobutene is selectively dimerized to form primarily branched C₈ olefins. Effluent from the dimerization reactors is fractionated in unit 111 to separate C₄ and lighter products in stream 112, with the dimer product passing to the aromatization zone 120 via line 113.

The aromatization zone converts the dimer product from zone 110 to yield a high proportion of para-xylene. The aromatization zone 120 typically uses a plurality of reactors arranged in series with the entire feed passing through each reactor. Since the aromatization reaction is highly endothermic, a series of reactors with reheating between each reactor permits greater control of the processing temperature. Hydrogen generated by aromatization is circulated within the aromatization zone, with a net hydrogen stream recovered via line 121. Liquid effluent from aromatization passes via line 122 to unit 123 to separate C₄ and lighter products in line 124. Debutanized product from 123 passes in line 125 to unit 126 for further separation of products. Unit 126 may be a sidestream fractionator as shown, or it may comprise two or more fractionating columns. In any event, a stream comprising C₅ to C₇ hydrocarbons is removed from the process in line 127 and a stream comprising C₉ and heavier hydrocarbons is removed in line 128 in order to provide a para-xylene concentrate 129 as feed to para-xylene-recovery zone 130.

Separation zone 130 may comprise any suitable process to recover para-xylene of the desired purity, usually >99.7% purity, from the concentrate in line 129, including without limitation one or more of continuous adsorption, pressure-swing adsorption, fractionation and crystallization. Single-stage crystallization is preferred as a relatively inexpensive technique to separate high-purity para-xylene in line 131 from a feed rich in the para-isomer. The reject stream in line 132 is rich in other C₈-aromatics isomers. Streams 127, 128 and 132 all are suitable components for gasoline blending, being rich in high-octane aromatics.

The feed to the aromatization process preferably is prepared by dimerization of isobutene. Suitable dimerization zones for this invention may be known by a variety of names and employ one or more of several catalyst types. Other names for the dimerization zone include oligomerization, catalytic condensation and catalytic polymerization. The use of resin catalysts for effecting dimer production is described, for example, in U.S. Pat. Nos. 4,100,220; 4,215,011; and 4,302,356. The use of a layered molecular sieve for isobutene oligomerization is taught in U.S. Pat. No. 6,649,802 B1. U.S. Pat. No. 6,689,927 B1 discloses oligomerization of isobutene using a solid phosphoric acid catalyst. The applicable teachings of all of the above references in this paragraph are incorporated herein by reference thereto. An effective dimerization zone provides a high yield of iso-octenes comprising a high concentration of 2,4,4-trimethylpentene in the product from the zone.

A dimerization catalyst preferably is disposed in fixed beds within the dimerization zone in what is known as a chamber-type reactor structure. In a chamber-type reactor, the reactants flow through one or more fixed catalyst beds. The temperature gradient within the reactor from the exothermic dimerization reaction is controlled by recycling relatively inert hydrocarbons which act as a heat sink. The unreacted isobutane from the dehydrogenation zone supplies a large proportion of the inert hydrocarbons that act as the heat sink. The temperature gradient within the dimerization reaction zone also may be controlled by the use of a quench material between the catalyst beds. As a secondary purpose, the quench material can provide a flushing function to inhibit the development of coke and the deactivation of coke in the deactivation of the catalyst within the reaction zones. Unconverted isobutene, containing unconverted butanes from the dehydrogenation zone, from stabilization of the butene dimer may be used as quench. Higher molecular weight quench material may be used within the dimerization reaction zones to flush the catalyst and preventing coke production. The recycle of such materials as the C₅to C₇ byproduct from the aromatization zone can also improve selectivity of the dimerization zone to produce the desired C₈ products. Since the higher molecular weight materials have benefits beyond use as a quench, it can be beneficial to add all or a portion of such material to the inlet of dimerization reactor with the feed.

A particularly preferred dimerization catalyst is a cationic resin catalyst such as the Amberlyst series (for example, Amberlyst 15) as produced by Rohm & Haas. The present process preferably is carried out in a substantially vertical fixed catalyst bed; for example, a bed of cation exchange resin supported in a vertical reactor. The flow in the reactor may be upward or downward, with downflow being preferred. Generally, the liquid hydrocarbon and an optional water, ether and/or alcohol cofeed may pass through a single line or separate lines into the reactor. A preferred cofeed concentration is an equivalent of 0.001 to 1 mol of t-butanol per mol of isobutene.

A range of yields may be effected by varying conversion, as illustrated by the following yields from a feedstock containing 43.5 wt.-% isobutene and 1.5 wt.-% normal butene with the balance being primarily butanes:

| | | | |
|---|---|---|---|
| Isobutene conversion, % | 49.6 | 68.1 | 83.3 |
| Hydrocarbon product distribution, wt.-%: | | | |
| C₇- | 0.26 | 0.46 | 0.44 |
| Di-isobutene | 90.0 | 84.4 | 82.5 |
| Other C₈ | 0.49 | 0.82 | 1.03 |
| C₉ and heavier (~80% tri-isobutene) | 9.25 | 14.3 | 16.0 |

The product also contained about 0.5 wt.-% ethers.

Preferred dimerization conditions when utilizing a resin catalyst comprise a liquid hourly space velocity (LHSV) with respect to isobutene of 0.1 to 3.0, with LHSV of 0.5 to 2.0 being preferred, based on fresh feed (i.e., excluding recycle). Reaction temperature generally ranges between 55° and 160° C., with a preferred temperature range of about 100° to 130° C. There may be a temperature gradient through the bed, which preferably is no greater than about 10° to 25° C. The reaction is carried out under sufficient pressure to maintain a liquid phase system, e.g., 1.5 to 2.5 MPa.

A well known alternative catalyst for the dimerization process is a lo solid phosphoric acid (SPA) catalyst. The SPA catalyst refers to a solid catalyst that contains as a principal ingredient an acid of phosphorus such as ortho-, pyro- or tetraphosphoric acid. The catalyst is normally formed by mixing the acid of phosphorus with a siliceous solid carrier to form a wet paste. This paste may be calcined and then crushed to yield catalyst particles where the paste may be extruded or pelleted prior to calcining to produce more uniform catalyst particles. The carrier is preferably a naturally occurring porous silica-containing material such as kieselguhr, kaolin, infusorial earth, and diatomaceous earth. A minor amount of various additives such as mineral talc, fuller's earth, and iron compounds including iron oxide may be added to the carrier to increase its strength and hardness. The combination of the carrier and the additives preferably comprises about 15-30% of the catalyst, with the remainder being the phosphoric acid. The additive may comprise about 3-20% of the total carrier material. Variations from this such as a lower phosphoric acid content are however possible. Further details as to the composition and production of SPA catalysts may be obtained from U.S. Pat. Nos. 3,050,472; 3,050,473; and 3,132,109 and from other references.

When utilizing the alternative SPA catalyst, dimerization conditions comprise a preferred temperature in the reaction zone of from about 90° to 260° C., and more typically in a range of from about 150° to 230° C. Pressures within the dimerization reaction zone will usually be in a range of from 200 kPa to 8 MPa, and more typically in a range of from 1.4 to 4 MPa. Steam or water may be fed into the reactor to maintain the desired water content in the preferred catalyst.

Effluent from the dimerization zone is stabilized to separate overhead unconverted isobutene along with butanes and lighter hydrocarbons. The stabilizer overhead may be recycled to the dimerization zone for further conversion of the isobutene as well as for temperature control of the reaction. The stabilized dimerization product, comprising one or both of iso-octenes and iso-octanes, comprises the feed to the aromatization zone.

It is within the scope of the present invention that part or all of the butene dimer is processed in a dimer hydrogenation zone before being passed to the aromatization zone. Suitable conditions and catalysts for dimer hydrogenation are taught in U.S. Pat. Nos. 5,847,252; 5,856,604 and 6,025,533, incorporated herein by reference thereto. The hydrogenation zone would yield a hydrogenated dimer-comprising 2,2,4-trimethylpentane and 2,5-dimethylhexane along with unconverted butene dimer as feed to the aromatization zone. This optional hydrogenation would also generate part if not all of the heat required to convert partially or fully hydrogenated butene dimer to aromatics. Preferably, however, the stabilized butene dimer is not fully hydrogenated before passing to the aromatization zone.

The butene dimer passing to the aromatization zone comprises a high concentration of one or more of 2,4,4-trimethylpentene, 2,2,4-trimethylpentane, 2,5-dimethylhexene and 2,5-dimethylhexane. The present process is particularly effective for the aromatization of butene dimer that is not fully hydrogenated, namely a feed stream containing some 2,4,4-trimethylpentene, which is less-readily converted in processes of the known art.

The aromatization process may be effected in a reactor section comprising one reactor or in multiple reactors with provisions known in the art to adjust inlet temperatures to individual reactors. The feed may contact the catalyst system in each of the respective reactors in either upflow, downflow, or radial-flow mode. Since the preferred aromatization process operates at relatively low pressure, the low pressure drop in a radial-flow reactor favors the radial-flow mode. As the predominant dehydrocyclization reaction is endothermic, the reactor section generally will comprise two or more reactors with interheating between reactors to compensate for the endothermic heat of reaction and maintain dehydrocyclization conditions.

The reactor section usually is associated with catalyst-regeneration options known to those of ordinary skill in the art, such as: (1) a semiregenerative unit containing fixed-bed reactors maintains operating severity by increasing temperature, eventually shutting the unit down for catalyst regeneration and reactivation; (2) a swing-reactor unit, in which individual fixed-bed reactors are serially isolated by manifolding arrangements as the catalyst become deactivated and the catalyst in the isolated reactor is regenerated and reactivated while the other reactors remain on-stream; (3) a moving-bed reactor with continuous catalyst withdrawal, regeneration, reactivation and substitution of the reactivated catalyst, permitting higher operating severity by maintaining high catalyst activity through regeneration cycles of a few days; (4) a hybrid system with semiregenerative and continuous-regeneration provisions in the same unit; (5) an ebullated-bed reactor with continuous catalyst withdrawal and regeneration; (6) a continuously stirred tank reactor; or (7) a riser-reactor reforming process, generally associated with a fluidized reactor and continuous catalyst regeneration according to U.S. Pat. No. 5,565,090 which is incorporated herein by reference. The preferred embodiment of the present invention is a moving-bed reactor with continuous catalyst regeneration.

An aromatization catalyst preferably incorporates porous, adsorptive, high-surface-area materials. Within the scope of the present invention are refractory supports containing one or more of: (1) refractory inorganic oxides such as alumina, silica, titania, magnesia, zirconia, chromia, thoria, boria or mixtures thereof, (2) synthetically prepared or naturally occurring clays and silicates, which may be acid-treated; (3) crystalline zeolitic aluminosilicates, either naturally occurring or synthetically prepared such as FAU, MEL, MFI, MOR, MTW, MWW (IUPAC Commission on Zeolite Nomenclature), in hydrogen form or in a form which has been exchanged with metal cations; (4) spinels such as MgAl₂ O₄, FeAl₂ O₄, ZnAl₂ O₄; and (5) combinations of materials from one or more of these groups.

The favored aromatization catalyst comprises a non-acidic large-pore molecular sieve. Suitable molecular sieves generally have a maximum free channel diameter or “pore size” of 6 Å or larger, and preferably have a moderately large pore size of about 7 to 8 Å, and materials with a significant amounts of external surface. Such molecular sieves include those characterized as LTL, BPH, OFF, MOR, MTW, FAU, AFI, BEA or MWW structure type by the IUPAC Commission on Zeolite Nomenclature, with the LTL structure being preferred. It is essential that the preferred L-zeolite be non-acidic, as acidity in the zeolite lowers the selectivity to aromatics of the finished catalyst. In order to be “non-acidic,” the zeolite has substantially all of its cationic exchange sites occupied by nonhydrogen species. Preferably the cations occupying the exchangeable cation sites will comprise one or more of the alkali and alkaline earth metals, particularly Li, Na, K, Rb, Cs, Mg, Ca, Sr and Ba. Other cationic species may be present alternatively or in addition to the foregoing. An especially preferred nonacidic L-zeolite is potassium-form L-zeolite.

A preferred molecular sieve utilized in the aromatization catalyst has a unit empirical formula on an anhydrous basis of:

mA:(S_{w} Alₓ Si_{y})O₂

where “A” is at least one exchangeable cation selected from the group consisting of alkali and alkaline earth metals, “S” is a metal comprising one or more of Sn, Zn, B, In, Ga and Ge, “m” is the mole fraction of A and varies from about 0.01 to about 0.49, “w” is the mole fraction of metal S and varies from about 0.01 to about 0.49, “x” is the mole fraction of aluminum and varies from about 0.01 to about 0.49 and “y” is the mole fraction of silicon and varies from about 0.50 to about 0.98. An especially preferred molecular sieve utilizes tin as the metal in the above formula, which then becomes:

mA:(Sn_{w} Alₓ Si_{y})O₂

The especially preferred molecular sieves are conveniently prepared by he technique disclosed in U.S. Pat. No. 5,518,708 which teachings are incorporated by reference. Generally, the process involves contacting a crystalline zeolite having a molar SiO₂/Al₂ O₃ ratio of at least 2 with an effective amount of a fluoro salt of tin, preferably in an amount of at least 0.0075 moles per 100 grams of zeolite starting material, the fluoro salt preferably being in the form of an aqueous solution or slurry which is contacted with the zeolite either incrementally or continuously at a slow rate (optionally in the presence of a buffer) whereby framework aluminum atoms of the zeolite are removed and replaced by tin atoms while retaining at least 80 percent and more preferably at least 90 percent of the crystal structure of the starting zeolite.

The fluoro salt preferably is provided as an aqueous solution or slurry, but solutions or slurries employing alcohols or other organic solvents may be suitable alternatives. An effective amount of fluoro salt is that amount which provides sufficient fluoride and tin for the process and the desired amount of tin in the final molecular sieve product. Solutions having fluoro salt concentrations of between about 10⁻³ moles per liter of solution and up to saturation of the solution can be employed, but preferably concentrations of between about 0.5 and about 1.0 moles per liter of solution are used. The minimum value for the amount of fluoro salt to be added is usually at least equivalent to the minimum mole fraction of aluminum to be removed from the zeolite.

The solution or slurry is maintained at an effective pH such that, under effective process conditions, a monomeric species of the tin is present in the reaction solution and the pH is high enough to avoid undue destructive acidic attack on the particular zeolite. The effective pH value generally is greater than 1, preferably greater than 3 and more preferably in the range of about 3 to about 7. Crystal degradation of many zeolites is found to be unduly severe at pH values below about 3, whereas insertion of the tin may be slow from a practical standpoint as a result of the solubility of tin at a pH of 7 and above.

The fluoro salt used as the aluminum extractant and as the source of tin can be any of the fluoro salts having the general formula:

A_{2/b} SnF₆ or A_{2/b} SnF₄

where “A” is a metallic or non-metallic cation having a valence “b” including alkylammonium, H⁺, NH₄⁺, Mg⁺⁺, Li⁺, Na⁺, K⁺, Ba⁺⁺, Cd⁺⁺, Cu⁺⁺, Ca⁺⁺, Cs⁺, Fe⁺⁺, Co⁺⁺, Pb⁺⁺, Mn⁺⁺, Rb⁺, Ag⁺, Sr⁺⁺, Tl⁺ and Zn⁺⁺. The ammonium and hydronium cation forms of the fluoro salt are generally preferred because of their solubility in water and also because these cations form water soluble by-product salts upon reaction with the zeolite, e.g., (NH₄)₃ AlF₆ and/or (NH₄)₂ AlF₅. Other salts which may be used include a combination of salts of SnF₂ and 3/2 (NH₄ HF₂) or SnF₄ and NH₄ HF₂. Preferred fluoro salts are NH₄ SnF₃; SnF₂^{.3}/2 (NH₄ HF₂) and SnF₄.NH₄ HF₂.

The preferred effective reaction temperature is between about 10° C. and about 99° C., preferably between about 20° and 95° C., but temperatures of 125° C. or higher and as low as 0° C. may be employed in some cases. Reaction temperature and reagent concentrations are optimized with respect to the zeolite starting material to provide adequate time for insertion of framework tin consistent with practical commercial considerations. Generally more highly siliceous zeolites enable higher permissible reaction temperatures and lower pH conditions.

In specifying the proportions of the zeolite starting materials or adsorption properties of the zeolite product and the like herein, the “anhydrous state” of the zeolite will be intended unless otherwise stated. The term “anhydrous state” is employed herein to refer to a material substantially devoid of both physically adsorbed and chemically adsorbed such as is typically obtained by heating the zeolite in dry air at about 450° C. for about 4 hours.

It is necessary to composite the zeolite with a binder in order to provide a convenient form for use in the catalyst particles of the present invention. The art teaches the suitability of a variety of refractory inorganic oxide binders. One or more of silica, alumina or magnesia are preferred binder materials of the present invention. One or both of amorphous silica and alumina are especially preferred. In one embodiment, excellent results are obtained when using a synthetic white silica powder precipitated as ultra-fine spherical particles from a water solution. A silica binder preferably is nonacidic, contains less than 0.3 wt.-% sulfate salts, and has a BET surface area of from about 120 to 160 m²/g.

The zeolite and binder may be composited to form particle shapes known to those skilled in the art such as spheres, extrudates, rods, pills, pellets, tablets or granules. The preferred form of a zeolite-containing aromatization catalyst is a cylindrical extrudate. In one method of forming extrudates, potassium-form L-zeolite and amorphous silica are commingled as a uniform powder blend prior to introduction of a peptizing agent. An aqueous solution comprising sodium or potassium hydroxide is added to form an extrudable dough. The dough preferably will have a moisture content of from 30 to 50 wt.-% in order to form extrudates having acceptable integrity to withstand direct calcination. The resulting dough is extruded through a suitably shaped and sized die to form extrudate particles, which are dried and calcined generally by known methods. Preferably, extrudates are subjected directly to calcination without an intermediate drying step in order to encapsulate potassium ions and preserve basicity. The calcination of the extrudates is effected in an oxygen-containing atmosphere at a temperature of from about 260° to 650° C. for a period of about 0.5 to 2 hours.

An essential ingredient of the aromatization catalyst is a metal component comprising at least one metal selected from Groups VIII (IUPAC 8-10) and IA (IUPAC 11) of the Periodic Table, including the platinum-group metals, Fe, Co, Ni, Cu, Ag and Au. Of the preferred Group VIII platinum-group metals, i.e., platinum, palladium, rhodium, ruthenium, osmium and iridium, platinum is particularly preferred. Mixtures of platinum-group metals as a uniformly distributed component or platinum-group surface metals also are within the scope of this invention. The platinum-group metal component may exist within the final catalytic composite as a compound such as an oxide, sulfide, halide, or oxyhalide, in chemical combination with one or more of the other ingredients of the composite, or as an elemental metal. Best results are obtained when substantially all of the metals are present in the elemental state. The platinum-group metal component may be present in the final catalyst composite in any amount which is catalytically effective, but relatively small amounts are preferred. The uniformly distributed platinum-group metals generally will comprise from about 0.01 to 5 wt.-% of the final catalyst, and preferably about 0.05 to 2 wt.-%, calculated on an elemental basis.

The preferred platinum-group metal component may be incorporated into the aromatization catalyst in any suitable manner such as coprecipitation or cogellation with the carrier material, ion exchange or impregnation. Impregnation using water-soluble compounds of the metal is preferred. Typical platinum-group compounds which may be employed are chloroplatinic acid, ammonium chloroplatinate, bromoplatinic acid, platinum dichloride, platinum tetrachloride hydrate, tetraamine platinum chloride, tetraamine platinum nitrate, dinitrodiaminoplatinum, platinum dichlorocarbonyl dichloride, palladium chloride, palladium chloride dihydrate, palladium nitrate, and the like. Chloroplatinic acid or tetraamine platinum chloride are preferred as the source of the preferred platinum component.

The aromatization catalyst may contain a halogen component. The halogen component may be either fluorine, chlorine, bromine or iodine or mixtures thereof with chlorine being preferred. Considering the nonacidic nature of the support, the halogen usually is incorporated into the catalyst only in association with the incorporation of a metal component. The halogen component is generally present in a combined state with the inorganic-oxide support. The halogen component is preferably well distributed throughout the catalyst and may comprise from more than 0.2 to about 15 wt.-% calculated on an elemental basis, of the final catalyst.

It is within the scope of the present invention that the aromatization catalyst may contain supplemental metal components known to modify the effect of the preferred platinum component. Such metal modifiers may include one or more of the Group IVA (IUPAC 14) metals, Group 1A (IUPAC 11) metals, rhenium, indium, gallium, bismuth, zinc, uranium, thallium and the rare-earth elements (lanthanide metals). Group VIB (IUPAC 6) metals are disfavored, considering the known toxicity of chromium. One or more of tin, germanium gallium, indium, copper, silver, gold, lead, zinc and the rare-earth elements are favored modifier metals, with tin, indium, germanium, cerium and lead being particularly favored. If present, the concentration of a metal modifier in the catalyst may be within the range of 0.001 to 5.0 wt.-%. Catalytically effective amounts of such metal modifiers may be incorporated into the catalyst by any means known in the art.

The final aromatization catalyst generally will be dried at a temperature of from about 100° to 320° C. for about 0.5 to 24 hours, followed by oxidation at a temperature of about 300° to 550° C. in an air atmosphere which preferably contains a chlorine component for 0.5 to 10 hours. Preferably the oxidized catalyst is subjected to a substantially water-free reduction step at a temperature of about 300° to 550° C. for 0.5 to 10 hours or more. The duration of the reduction step should be only as long as necessary to reduce the platinum, in order to avoid pre-deactivation of the catalyst, and may be performed in-situ as part of the plant startup if a dry atmosphere is maintained.

The dimerization product stream contacts the aromatization catalyst in the aromatization zone at aromatization conditions to obtain an aromatized effluent, with the principal reaction being dehydrocyclization of olefinic and paraffinic hydrocarbons to obtain xylenes having a higher-than-equilibrium concentration of para-xylene. Aromatization conditions include a pressure of from about 100 kPa to 6 MPa (absolute), with the preferred range being from 100 kPa to 1 MPa (absolute) and a pressure of about 450 kPa or less at the exit of the last reactor being especially preferred. The volume of the contained aromatization catalyst corresponds to a liquid hourly space velocity of from about 1 to 40 hr⁻¹. Free hydrogen as molecular H₂ is supplied to the aromatization zone in an amount sufficient to correspond to a ratio of from about 0.1 to 10 moles of hydrogen per mole of hydrocarbon feedstock; other components of a hydrogen-containing gas stream may comprise one or more of hydrocarbons, nitrogen and steam. The operating temperature, defined as the maximum temperature of the combined hydrocarbon feedstock, free hydrogen, and any components accompanying the free hydrogen, generally is in the range of 260° to 560° C. Hydrocarbon types in the feed stock also influence temperature selection.

The aromatization process produces an aromatics-rich effluent stream, with the aromatics content of the C₅+ portion of the effluent typically within the range of about 45 to 95 wt.-%, and more usually more than about 85 wt.-%. The composition of the aromatics will depend principally on the feedstock composition and operating conditions. From the present dimerized isobutene feedstock, the aromatics consist principally of C₈ aromatics with a high para-xylene content.

Using techniques and equipment known in the art, the aromatics-rich effluent from the aromatization zone usually is passed through condensing and cooling facilities to a separator. A hydrogen-rich gas is separated and recycled through suitable compressing means to the first reactor of the aromatization zone, with some net hydrogen available for other uses. The liquid phase from the separation zone is normally withdrawn and processed in a fractionating system.

The aromatization product is fractionated by conventional means to separate C₄ and lighter materials, which may be returned to the light-ends processing section of the dimerization zone in order to recycle butanes to the deisobutanizer. C₅ to C₇ hydrocarbons are removed by fractionation for blending into gasoline or processing in conventional refining units for recovery of benzene and toluene values. Mixed C₈ aromatics, representing a para-xylene concentrate, are recovered overhead in a rerun column, with the bottoms stream, comprising C₉ and heavier aromatics, being a desirable component for blending into premium gasoline. Optionally, the para-xylene concentrate is separated from C₅ to C₇ hydrocarbons and C₉ and heavier aromatics in a sidestream fractionator.

The xylene yield relative to conversion of dimerization product in the aromatization zone generally is at least about 25 wt.-%, and more usually 35 wt.-% or more. A xylene yield of about 40 to 50 wt.-% or more often is attainable in the present process.

In conjunction with the above xylene yields, the concentration of para-xylene in xylenes as represented by the para-xylene concentrate will be significantly above the equilibrium value of 20 to 25 wt.-%. Para-xylene concentration in the xylenes usually will be about 40 wt.-% or more, and often at least about 50 wt.-%. Concentrations of about 60 wt.-% or more of para-xylene in xylenes are achievable, and a concentration of about 85 wt.-% enables ready use of single-stage crystallization for para-xylene recovery.

At least a portion of the para-xylene concentrate is passed to the para-xylene purification zone. This zone comprises any suitable process for recovering high-purity para-xylene product. Suitable processes may include one or more of crystallization, simulated-moving-bed adsorption, pressure-swing adsorption and fractionation. An integrated adsorption and crystallization process is described in U.S. Pat. No. 5,329,060, the provisions of which are incorporated herein by reference. Crystallization, and especially single-stage crystallization, is preferred for para-xylene separation from the para-xylene concentrate of the present invention.

Para-xylene recovery by crystallization from mixed C₈ aromatics is well known. U.S. Pat. Nos. 2,866,833 and 2,985,694 describe multi-stage crystallization processes for para-xylene recovery. Such processes have the disadvantage of low para-xylene recovery due to the formation of eutectic binaries in the mother liquor from which the para-xylene crystals are recovered as well as high operating costs resulting from the multiple stages. U.S. Pat. No. 5,319,060 teaches overcoming this disadvantage by using selective adsorption to enrich the para-xylene feed to crystallization, enabling the use of single-stage crystallization. The relevant contents of the above patents are incorporated herein by reference thereto.

Feed generally enters a crystallizer near the top and exits near the bottom. Each crystallizer is usually equipped with scrapers that remove crystals adhering to the internal walls of the vessel. Crystallizer slurry can be recirculated to the crystallizer to classify the crystals within the crystallizer. effluent from the crystallizer is passed to a centrifuge, which operates to separate the mother liquor from the para-xylene crystals.

Since the concentration of para-xylene in the mixed C₈ aromatics is relatively high, generally in excess of 70 wt.-% and more usually at least about 85 wt.-%, a purification zone comprising crystallization usually can be reduced to a single stage. This stage can be operated at purification conditions approximating those of the final stage of multi-stage crystallization, for example, temperatures of 0 to −10° C. Chilling usually can be provided by propane vaporization. The crystallization is limited only by the amount of solids that can readily flow in a stream rather than the previously mentioned eutectic limit. At least a portion of the mother liquor can be recycled and mixed with the crystallization feed to provide more liquor to carry additional recovered para-xylene, with the remaining net portion being a desirable component for blending into premium gasoline. Alternatively or in addition, additional para-xylene can be recovered from the mother liquor which may comprise an above-equilibrium concentration of para-xylene.

Optional additional treatment of the stage para-xylene crystals may include washing the crystals with a variety of compounds including but not limited to para-xylene product, normal pentane, toluene, aqueous alcohols and aqueous salts to improve final product purity by removing adhering second stage mother liquor. After melting the crystals, it may be necessary to feed the resulting mixture to a fractionation column to separate the para-xylene product from the wash liquor.

The high-purity para-xylene recovered from the purification zone comprises at least about 99.5 wt.-% para-xylene, and preferably at least about 99.7 wt.-% para-xylene.

Other embodiments and variants encompassed by and within the spirit of the present invention as claimed will be apparent to the skilled routineer. Examples follow which illustrate certain specific embodiments, and these particularly should not be construed to limit the scope of the invention as set forth in the claims.

### EXAMPLES

### Example 1

A support prepared according to the known art, comprising 80 wt.-% K-L and 20 wt.-% alumina, was finished with tetraamineplatinum chloride (TAPC) and KCl to give 1.0 wt.-% Pt and 15 wt.-% K. This catalyst is designated as Catalyst Z.

### Example 2

Ammonium-exchanged zeolite L was slurried in distilled water and heated to 75° C. A solution containing SnF₄ and NH₄ HF₂ in distilled water was added incrementally over a period of 10 minutes to the zeolite. Following the addition of the tin solution, the slurry was digested over a period of 1.5 hours and the product was filtered and washed free of soluble fluoride. The solid product (NH₄—Sn-L) was white and showed the characteristic crystal structure of zeolite L as indicated by X-ray powder diffraction.

The NH₄—Sn-zeolite L solid was slurried in distilled water, and the slurry was stirred and heated. KCl then was added and the mixture was allowed to react for 1-1.5 hours hours, filtered, and the filtrate was washed with deionized water. This procedure was repeated 5 times, and the material was then calcined and calcined. This sample had the following metals contents: 0.57% Sn, 9.38% Al, 30.2% Si, and 13.7% K.

The calcined K—Sn-L was platinum impregnated using tetraamineplatinum chloride (TAPC) by evaporative impregnation to give a catalyst containing 0.39 wt.-% Pt and 0.57 wt.-% Sn. This catalyst is designated as Catalyst A.

### Example 3

A similar catalyst was prepared following the procedure described in Example 2 to give 0.30 wt.-% Pt and 0.33 wt.-% Sn. This catalyst is designated as Catalyst B.

### Example 4

The catalyst is prepared following the procedure described in Example 2 to give 0.60 wt.-% Pt and 0.33 wt.-% Sn. This catalyst is designated as Catalyst C.

### Example 5

Catalysts were pelletized into 20×40 mesh size and evaluated in a micro-reactor unit at atmospheric pressure. The catalysts were pre-reduced in H₂ flow at 450° C. for 2 hours. The reactor then was cooled to 300° C. and hydrogen flow was directed through a 2,4,4-trimethylpentene bath and into the reactor. The catalyst performance is then collected at 500 and 550° C. based on data from non-polar and polar GC columns. The results as indicated in the Table show that catalysts A, B and C of the invention provide a combination of high xylene yields and high para-xylene content of the xylene product. Catalyst Z of the known art did not achieve a combination of 25% or higher xylene yield relative to conversion and about 50% or higher para-xylene in the xylene product, levels which were achieved by all of the catalysts of the invention.

TABLE

| Catalyst | | Temp. | Conversion | Xylenes | B/T/EB | C₁-C₇ | C₄+ = C₄ | Xylenes/ | P-xylene/ |
|---|---|---|---|---|---|---|---|---|---|
| Desig. | mg | ° C. | wt.-% | wt.-% | wt.-%^{#} | wt-%* | wt.-%^{&} | Conversion | Xylenes |
| Z (art) | 1000 | 500 | 62.04 | 25.12 | 38.54 | 22.52 | 8.71 | 40.5% | 36.1% |
| Z (art) | 1000 | 550 | 99.86 | 28.98 | 76.94 | 22.31 | 9.91 | 29.0% | 33.8% |
| A (inv.) | 1000 | 500 | 68.21 | 21.31 | 25.48 | 35.75 | 23.74 | 31.2% | 51.0% |
| A (inv.) | 1000 | 550 | 87.07 | 40.34 | 46.17 | 37.31 | 29.38 | 46.3% | 56.0% |
| B (inv.) | 1000 | 500 | 32.44 | 8.99 | 9.67 | 15.21 | 9.24 | 27.7% | 60.4% |
| B (inv.) | 1000 | 550 | 65.34 | 31.22 | 33.58 | 24.33 | 17.17 | 47.8% | 60.4% |
| C (inv.) | 1000 | 500 | 64.40 | 26.54 | 29.08 | 23.97 | 12.51 | 41.2% | 49.0% |
| C (inv.) | 1000 | 500 | 82.27 | 44.17 | 50.42 | 25.65 | 16.00 | 53.7% | 51.9% |

^{#}BTX is benzene, toluene and ethylbenzene
*C₁ to C₇ paraffins and olefins
^{&}butanes + butenes, included in C₁-C₇

We claim:

1. A process combination for the production of high-purity para-xylene from 2,4,4-trimethylpentene comprising contacting a butene dimer comprising 2,4,4-trimethylpentene with a non-acidic large-pore molecular-sieve catalyst in an aromatization zone operating at aromatization conditions to produce a para-xylene concentrate comprising xylenes having a higher-than-equilibrium content of para-xylene.

2. The process of claim 1 wherein the aromatization conditions comprise a pressure of from about 100 kPa to 6 MPa (absolute), a hydrogen to hydrocarbon ratio of from about 0.1 to 10, a liquid hourly space velocity of from about 1 to 40 hr⁻¹, and an operating temperature of from about 260° to 560° C.

3. The process of claim 1 wherein the aromatization catalyst comprises:
(a) a nonacidic large-pore molecular sieve;
(b) a hydrogenation metal selected from one or more of the platinum-group metals;
(c) a metal modifier selected from one or more of tin, indium, germanium, gallium, copper, silver, gold, lead, zinc and the rare-earth elements.

4. The process of claim 3 wherein the molecular sieve utilized in the aromatization catalyst suitably has a unit empirical formula on an anhydrous basis of mA:(Sn_{w} Alₓ Si_{y})O₂ where A is at least one exchangeable cation selected from the group consisting of alkali and alkaline earth metals, “m” is the mole fraction of A and varies from about 0.01 to about 0.49, “w” is the mole fraction of tin and varies from about 0.01 to about 0.49, “x” is the mole fraction of aluminum and varies from about 0.01 to about 0.49 and “y” is the mole fraction of silicon and varies from about 0.50 to about 0.98.

5. The process of claim 3 wherein the aromatization catalyst comprises the substantial absence of a Group VIB (6) metal.

6. The process of claim 3 wherein the catalyst contains about 0.01 to 5.0 wt % platinum.

7. The process of claim 1 wherein the content of para-xylene in the para-xylene concentrate is at least about 40 wt.-%.

8. The process of claim 1 wherein the content of para-xylene in the para-xylene concentrate is at least about 50 wt.-%.

9. A process combination for the production of high-purity para-xylene from a butene dimer comprising 2,4,4-trimethylpentene, comprising:
(a) contacting the butene dimer with an aromatization catalyst in an aromatization zone operating at aromatization conditions to produce a para-xylene concentrate comprising xylenes having a higher-than-equilibrium content of para-xylene and,
(b) passing at least a portion of the para-xylene concentrate to a para-xylene purification zone operating at purification-zone conditions to recover high-purity para-xylene.

10. The process combination of claim 9 wherein the aromatization catalyst comprises:
(a) a nonacidic large-pore molecular sieve;
(b) a hydrogenation metal selected from one or more of the platinum-group metals;
(c) a metal modifier selected from one or more of tin, indium, germanium, gallium, copper, silver, gold, lead, zinc and the rare-earth elements.

11. The process combination of claim 10 wherein the molecular sieve utilized in the aromatization catalyst suitably has a unit empirical formula on an anhydrous basis of mA:(Sn_{w} Alₓ Si_{y})O₂ where A is at least one exchangeable cation selected from the group consisting of alkali and alkaline earth metals, “m” is the mole fraction of A and varies from about 0.01 to about 0.49, “we is the mole fraction of tin and varies from about 0.01 to about 0.49, “x” is the mole fraction of aluminum and varies from about 0.01 to about 0.49 and “y” is the mole fraction of silicon and varies from about 0.50 to about 0.98.

12. The process combination of claim 10 wherein the aromatization catalyst comprises the substantial absence of a Group VIB (6) metal.

13. The process combination of claim 10 wherein the catalyst contains about 0.01 to 5.0 wt.-% platinum.

14. The process combination of claim 9 wherein the content of para-xylene in the para-xylene concentrate is at least about 40 wt.-%.

15. The process combination of claim 9 wherein the high-purity paraxylene comprises at least about 99.7 wt.-% para-xylene.

16. A process combination for the production of high-purity para-xylene from an isobutene-rich feed comprising:
(a) contacting the isobutene-rich feed with a dimerization catalyst in a dimerization zone operating at dimerization conditions to produce a butene dimer comprising 2,4,4-trimethylpentene;
(b) contacting at least a portion of the butene dimer with an aromatization catalyst in an aromatization zone operating at aromatization conditions to produce an para-xylene concentrate comprising xylenes having a higher-than-equilibrium content of para-xylene and,
(c) passing at least a portion of the para-xylene concentrate to a para-xylene purification zone operating at purification-zone conditions to recover high-purity para-xylene.

17. The process combination of claim 16 wherein the dimerization catalyst of step (a) comprises a cationic resin.

18. The process combination of claim 16 wherein the dimerization catalyst of step (a) comprises solid phosphoric acid.

19. The process combination of claim 16 wherein the aromatization catalyst comprises:
(a) a nonacidic large-pore molecular sieve;
(b) a hydrogenation metal selected from one or more of the platinum-group metals;
(c) a metal modifier selected from one or more of tin, indium, germanium, gallium, copper, silver, gold, lead, zinc and the rare-earth elements.

20. The process combination of claim 16 wherein the molecular sieve utilized in the aromatization catalyst suitably has a unit empirical formula on an anhydrous basis of mA:(Sn_{w} Alₓ Si_{y})O₂ where A is at least one exchangeable cation selected from the group consisting of alkali and alkaline earth metals, “m” is the mole fraction of A and varies from about 0.01 to about 0.49, “w” is the mole fraction of tin and varies from about 0.01 to about 0.49, “x” is the mole fraction of aluminum and varies from about 0.01 to about 0.49 and “y” is the mole fraction of silicon and varies from about 0.50 to about 0.98.
